(19) United States
(12) Patent Application Publication
(10) Pub. No.: US 2020/0147453 A1
(43) Pub. Date: May 14, 2020

(54) METHOD AND ELECTRONIC SYSTEM FOR SUPPORTING A PERSON IN THE PURSUIT OF SKI SPORT

(71) Applicant: ATOMIC Austria GmbH, Altenmarkt im Pongau (AT)

(72) Inventors: Jason ROE, Altenmarkt im Pongau (AT); Helmut HOLZER, Altenmarkt im Pongau (AT)

(73) Assignee: ATOMIC Austria GmbH, Altenmarkt im Pongau (AT)

(21) Appl. No.: 16/674,374

(22) Filed: Nov. 5, 2019

(30) Foreign Application Priority Data
Nov. 13, 2018 (AT) A 50983/2018

(51) Int. Cl. A63B 24/00; G09B 19/00; A61B 5/11; A43B 3/00

(52) U.S. Cl. CPC A63B 24/0062; A63B 24/0075; A63B 24/0006; G09B 19/0038; A61B 5/1124; A63B 2244/19; A63B 2024/0012; A63B 2024/0068; A63B 2220/51; A63B 2225/20; A61B 2503/10; A43B 3/0005

(57) ABSTRACT

A method and an electronic system for supporting a person in the pursuit of ski sport detects the current athletic performance ability or skill values of the person exercising sports using an electronic detection system that includes a portable mobile terminal, in particular a smartphone. An evaluation of the condition information or actual data provided by the electronic detection system using a computer-implemented evaluator and a determination of a current level of performance ability or skill of the person are carried out. Then, a selection or preselection of instructional material from instructional materials accessible or available to the computer-implemented evaluator is carried out by the computer-implemented evaluator taking into consideration or account the level of performance ability or skill of the person determined automatically or semi automatically. Automatically or semi-automatically selected instructional material is then immediately output on the mobile terminal portable by the person exercising sports.

### CROSS REFERENCE TO RELATED APPLICATIONS

Applicant claims priority under 35 U.S.C. § 119 of Austrian Application No. A 50983/2018 filed on Nov. 13, 2018, the disclosure of which is incorporated by reference.

### BACKGROUND OF THE INVENTION

### 1. Field of the Invention

The invention relates to a method for supporting a person, in particular an athlete, in the pursuit of ski sport. This method is at least partially carried out in a computer-implemented manner, meaning that it is implemented involving computers and/or software-controlled arithmetic units. The invention further relates to an electronic system for implementing the method according to the invention.

### 2. Description of the Related Art

AT517933B1, which traces back to the applicant, describes a sports boot for skiing as well as an electronic control system used in this context. This sports boot comprises an inner boot intended to receive the foot of a user, an outer shell of relatively hard and dimensionally stable plastic material surrounding at least sections of the inner boot on its outside, and at least one pressure-sensitive sensor for detecting mechanical pressures or forces in or on the sports boot. The sports boot further comprises a radio communication interface which is designed for wireless transmission of pressure signals or corresponding data from the at least one pressure-sensitive sensor. This radio communication interface on the sports boot is compatible with a radio communication interface on at least one electronic evaluation device. The electronic evaluation device is provided for evaluating the pressure signals detected by the at least one pressure-sensitive sensor and the pressure conditions in or on the sports boot derived therefrom. In the context of the electronic control system, a so-called digital skiing coach is to be created, with it being intended for said skiing coach and/or said electronic control system to support the user in optimizing the pursuit of the corresponding ski sport and/or make it more convenient. However, the results that can be achieved with the presented measures are satisfactory in partial aspects only.

WO2007/015908A2 describes a system for displaying the athletic running performance of a runner on electronic devices. At this, the signals of a step sensor in the sole of the running shoe, of a heart rate and/or blood pressure sensor on the body of the runner, and other sensor data are wirelessly transmitted to an electronic adapter device. In this regard, the adapter device, which receives at least the step signals, can be mechanically coupled and decoupled to the data interface of a standard display device via a plug and transmits the data to be visualized by the display device via this data interface. The adapter device and the display device, which can be formed by a standard mobile phone, a PDA, an MP3 player, a wristwatch and the like, are worn on the runner's body and are intended for evaluations of the runner's running performance. This known system is only suitable to a limited extent in connection with the pursuit of ski sport.

DE9417953U1 describes an insole for a ski boot. This insole is to serve as a learning aid for the performance of the correct swing. It consists of a flat and flexible insert covering the heel region. A cell filled with a liquid is arranged in this insert, wherein a sensor, which is connected to a power supply and a controller outside the ski boot via cables, reacting to pressure is arranged in the cell. The controller is in turn connected to a device for generating acoustic signals, in particular a stereo headset. This device is only partially satisfactory as an aid in the pursuit of ski sport.

### SUMMARY OF THE INVENTION

It was the object of the present invention to overcome the shortcomings of the prior art and to provide skiers with a technical equipment allow for an increase of the comfort and/or the performance that can be achieved with respect to the pursuit of sport.

This object is achieved by means of a method and an electronic system according to the invention.

The method according to the invention, which is in particular carried out in a computer-implemented manner, for supporting a person exercising sports, in particular an athlete, in the context of the pursuit of ski sport comprises the steps:
detecting the current and/or present athletic performance ability or skill values of the person exercising sports by means of an electronic detection system, said electronic detection system comprising a mobile terminal, in particular a smartphone or another wearable computer, portable by the person;
evaluating the condition information or actual data provided by the detection system by means of a computer-implemented evaluation means;
fully automated ascertainment or semi automated determination of the current level of performance capacity or skill of the person by means of the computer-implemented evaluation means;
selection or preselection of instructional material, for example of video-based training material, by means of the computer-implemented evaluation means from a plurality of instructional materials accessible or known to, in particular available to, the computer-implemented evaluation means, wherein this selection or preselection is carried out by the computer-implemented evaluation means taking into consideration or account the level of performance ability or skill of the person determined automatedly or semi automatedly;
outputting, in particular displaying, playing and/or rendering selected instructional material, in particular in the form of images and/or sound signals, directly on the mobile terminal portable by the person exercising sports.

An advantage of the measures according to the invention consists in that a high quality of support service can be achieved for the person exercising sports. To start with, a continuous and/or highly topical detection of the respective performance and/or skill values of the person exercising sports can be achieved by means of the electronic detection system. Such an electronic current state detection can yield comparatively objective results potentially even varying depending on the day. In particular, the detection of performance and/or skill values of the person exercising sports by means of the measures according to the claims is not entirely dependent on a subjective assessment and/or evaluation of the person exercising sports or another third person. In addition to this, by means of the computer-implemented evaluation means, training and/or instructional material as optimized as possible—for example in the form of video clips, audio books, commented picture series, text and/or voice instructions—is available relatively promptly and hence, an increase learning and/or training effect can be achieved. The training and/or instructional material selected or preselected depending on the current performance ability or skill values of the person can be provided either automatedly or with a user interaction as low as possible, i.e. semi automatedly, whereby time savings and simultaneous comfort enhancements can be achieved for the person exercising sports. Furthermore, the person exercising sports and/or the user of the specified system can be offered support as made-to-measure as possible by means of the at least semi automatedly selected training and/or instructional materials. In addition to this, the respective assistance can be used at almost any location and at any time that appears suitable, whereby the learning and training success for the person exercising sports can be increased even more.

In particular, it can be useful if an automated selection or preselection of for example video-base instructional material is provided for, wherein said instructional material can contribute to the improvement of the current athletic performance ability or skill values of the person exercising sports, i.e. the user of the system. Hence, the respective instructional material can be determined in a targeted manner, as error-free as possible and promptly and can then be provided to the person exercising sports.

According to an advantageous measure, displaying video images and/or text-based comments on a display of the portable mobile terminal can be provided for. In the alternative or in combination to this, an output of voice instructions by means of a speaker of the portable mobile terminal can be provided for. Hence, the costs for creating the specified technical system can be kept low. In particular, the development of a proprietary electronic output hardware is not required, whereby a high use acceptance can be achieved and/or a short learning and/or familiarization phase for users of the specified system can be attained.

In addition to this, it can be provided for that video-based instructional material is retrieved via a first data or communication interface of the portable mobile terminal, said data or communication interface being designed for remote transmission and remote reception of data in relation to at least one host computer or Internet portal. At this, the data interface can be designed according to the WLAN standard or according to a sufficiently powerful mobile network standard. Hence, the offer and/or volume of instructional material can be steadily extended and/or adapted without periodical or demand-based updates on the mobile terminal being required. Moreover, thereby a high volume of instructional material can be selected without local storage resources of the mobile terminal being impacted.

An embodiment, according to which graphics, symbols or information texts superimposed on a video-base instructional material in a representational manner, wherein this superimposition is carried out depending on execution deficits of the person to be supported determined automatedly, is also advantageous. Thereby, targeted and specific support of the athlete for the avoidance of certain and/or repeated execution errors can be achieved.

According to a further embodiment, a computer-implemented determination and output of a fatigue quantification and/or break recommendation on the portable mobile terminal can be provided for. Thereby, personal safety of the person exercising sports themselves, but also of other persons located in the immediate vicinity can be improved. In particular, gradual fatigue can be determined as objectively and/or quantitatively as possible by the electronic detection system and the person exercising sports and/or another user of the system, for example a trainer, can be made aware of gradual fatigue and the associated risks. Moreover, trend analyses and performance capacity developments can thereby be carried out.

It can further be useful if an output of visually, acoustically or tactilely perceptible signals by means of the display, the speaker and/or the vibration generator of the portable mobile terminal is carried out. Hence, the sensory perception of the person exercising sports respectively appearing most suitable can be used to interact and/or to reliably provide the person exercising sports with relevant information and/or statuses. Moreover, the hardware costs for implementing the specified system and/or method can be kept as low as possible by using standard-conform and/or common programmable terminals.

In addition to this, it can be provided for that parameters selected from the group comprising pursuit duration, speed profile, acceleration profile, force profile, sports equipment edging angle, ski style, weather conditions such as cold, warm, fog, snowfall and the like, piste conditions such as deep snow, hard, soft, icy, grippy, and piste difficulty levels such as blue, red or black category and off-piste, are included in the computer-implemented determination of a fatigue quantification and/or break recommendation.

Moreover, storing of detected performance ability or skill values and/or of determined levels of performance ability or skill in a local storage device of the portable mobile terminal and/or in an external remotely accessible storage device can be provided for. Hence, the sport- and exercise specific data can be reliably collected and kept ready for later evaluations and retrospective tasks.

According to a further embodiment, temporally preceding performance ability or skill values and/or determined levels of performance ability or skill stored in a storage device can be compared to current performance ability or skill values and/or determined levels of performance ability or skill by means of the computer-implemented evaluation means. In particular, relatively detailed trend analyses, by means of which the respective development of the person exercising sports can be well evaluated or traced, can thereby be allowed for.

Moreover, a comparison of performance ability or skill values and/or determined levels of performance ability or skill with respect to the persons belonging to a predefined comparison group can be provided for. Such a comparison can, for example, be made with a professional athlete, for example a World Cup participant, or with any person from a community. In particular, this can create a competition effect and/or a competition incentive, which can potentially further increase the sporting performance and/or motivation of persons exercising sports.

The electronic detection system can, according to an advantageous embodiment, comprise at least one pressure detection sensor, which is arranged in or on a sports boot than can be worn by the person. In this regard, at least one electronic evaluation unit can be provided, which evaluates the electrical pressure signals of the at least one pressure detection sensor or carried out a preprocessing or conditioning of the electrical pressure signals. Hence, profound conclusions on the movement behavior of the person exercising sports can be achieved without a complex compensation of a drift behavior of the at least one pressure detection sensor being required. Moreover, a corresponding pressure detection system can be implemented in or on the sports boot in a relatively robust and long-term stable way.

The electronic evaluation unit can be a component and/or a partial component of the electronic detection system. The electronic evaluation unit can be designed for being mounted preferably to at least one sports boot of the person exercising sports or to be carried at another position of the person exercising sports.

The object of the invention is further achieved by means of an electronic system according to the invention. The technical and advantageous effects that can thereby be achieved can be gathered from the above and below parts of the description.

### BRIEF DESCRIPTION OF THE DRAWINGS

Other objects and features of the invention will become apparent from the following detailed description considered in connection with the accompanying drawings.

It is to be understood, however, that the drawings are designed as an illustration only and not as a definition of the limits of the invention.

In the drawings,

FIG. 1 shows an exemplary embodiment of an electronic system for supporting a person in the pursuit of ski sport;

FIG. 2 shows a block diagram of an exemplary embodiment of an electronic, computer-implemented system for selecting or preselecting optimized or suitable instructional material for a person exercising sports; and

FIG. 3 shows a block diagram of an exemplary embodiment of an electronic, computer-implemented system for supporting a person in the pursuit of ski sport.

### DETAILED DESCRIPTION OF PREFERRED EMBODIMENTS

First of all, it is to be noted that in the different embodiments described, equal parts are provided with equal reference numbers and/or equal component designations, where the disclosures contained in the entire description may be analogously transferred to equal parts with equal reference numbers and/or equal component designations. Moreover, the specifications of location, such as at the top, at the bottom, at the side, chosen in the description refer to the directly described and depicted figure and in case of a change of position, these specifications of location are to be analogously transferred to the new position.

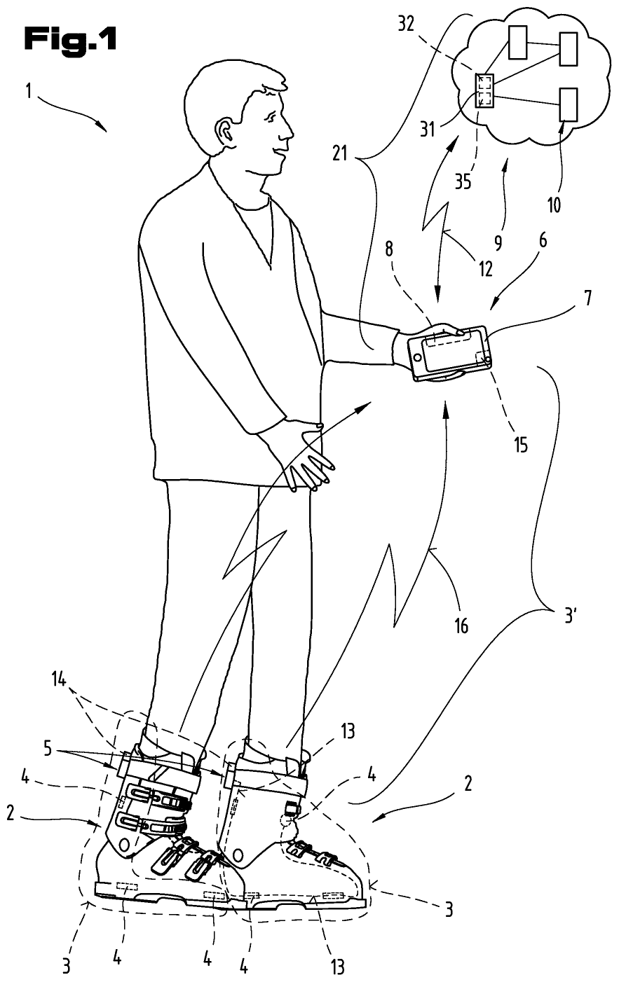

FIG. 1 shows a person 1 wearing and/or using technical equipment objects for exercising ski sports. Instead of the sports boot equipment for the pursuit of alpine ski sport shown by way of example, the system presented below can also be applied and/or used in the context of exercising cross-country skiing, snowboard skiing or touring skiing.

The person 1 shown in FIG. 1, who is equipped for the pursuit of alpine ski sport in accordance with the example, is supported in the pursuit of the corresponding sport by the system and method described below and/or coached and/or trained by the technical means described. The person 1 wears a pair of sports boots 2, alpine ski boots in accordance with the example, which can be coupled to and decoupled from a pair of skis, alpine skis (not shown) in accordance with the example, as required.

At least one of the sports boots 2, preferably both sports boots of the pair, comprise at least partial components of an electronic detection system 3, 3′. It is useful if the electronic detection system 3, 3′ comprises at least one pressure detection sensor 4 in and/or on the at least one sports boot 2 that can be worn by the person, at least one electronic preprocessing and/or evaluation unit 5 for the pressure signals of the at least one pressure detection sensor 4 and a mobile terminal 6 portable by the person 1. It is useful if the mobile terminal 6 is formed by a standard and/or common smartphone (cellphone) 7. This particularly applies where the mobile terminal 6 is assigned to the person 1 exercising sports and/or is to be ported by the person during the pursuit of sports. However, the mobile terminal 6 can also be formed by and/or comprise a tablet computer. This particularly applies where a comparatively large display surface is useful. Such a mobile terminal 6 can usefully be assigned to a trainer and/or to a coach additionally supporting the person 1 exercising sports. Especially the portable mobile terminal 6, which is assigned to the person 1 exercising sports, should be defined by a wearable computer, for example by a smartphone 7, by a smartwatch and/or by smartglasses (data glasses).

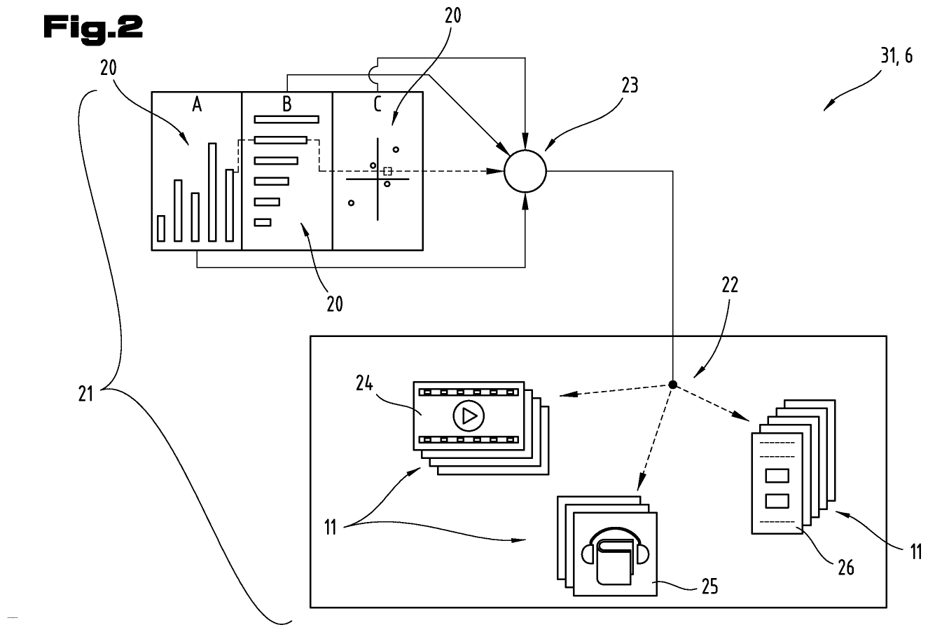

The respective mobile terminal 6 comprises at least one first communication interface 8 for data-technical communication with the Internet 9 and/or with a remote computer system 10. This first data-technical communication interface 8 is formed for bidirectional data transmission, and is in particular provided for downloading extensive information and/or comprehensive instructional material 11—FIG. 2. The first data and/or communication interface 8 is thus preferably adapted for setting up an Internet data connection 12 and can be defined by a standard data communication interface 8 of a smartphone 7. Accordingly, here, a wireless data transmission path and/or communication interface 8 is provided for.

The at least one electronic evaluation unit 5 for the pressure and/or force signals provided by the at least one pressure sensor 4 can—as is schematically depicted—be positioned on the sole side or be carried by the person 1 at another position. However, the electronic evaluation unit 5 can also be omitted. This particularly applies where the complete signal processing is carried out directly by the mobile electronic detection system 3′, in particular by the smartphone 7 of the person 1 and by the at least one pressure detection sensor 4. In this case, the pressure detection sensors 4 in and/or on the sports boot 2 each have radio communication interfaces for data and/or signal transmission to the mobile terminal 6. In particular, in this case the pressure detection sensors 4 are adapted for transmitting the respective pressure and/or force values directly to a wireless close-range communication interface 15 of the mobile terminal 6 and/or the smartphone 7.

However, according to a useful embodiment it is provided for that the pressure detection sensors 4 on and/or in the sports boot 2 transmit the respective electrical signals to the electronic preprocessing and/or evaluation unit 5 positioned on the side of the boot and/or of the person via electrical line links 13. Preferably, at least one signal-technical preprocessing and/or compression and data-technical processing of the pressure signals and/or pressure values, which are detected by the plurality of pressure detection sensors 4, is then carried out in the electronic evaluation unit 5. It is useful if at least two, preferably four or more, pressure detection sensors arranged so as to be distributed across the surface areas of the sports boot 2 are provided.

The electronic evaluation unit 5 that is preferably provided pair-wise, i.e. for each sports boot 2, in each case comprises at least one close-range radio communication interface 14. These close-range communication interfaces 14 are provided for data and/or signal-technical communication with a corresponding data-technical close-range communication interface 15 on the mobile terminal 6. These close-range communication interfaces 14, 15 can for example be realized according to the WLAN, RFID, Bluetooth or Zigbee standards, or be implemented according to another standardized data-technical communication technology. Via these corresponding close-range communication interfaces 14, 15, the signal values and/or data of the pressure detection sensors 4 can be wirelessly transmitted to the mobile terminal 6 via at least one schematically depicted close-range communication path 16.

At least by means of these pressure signals from the pressure detection sensors 4, evaluation conclusions regarding the use behavior and/or conclusions regarding the performance in the pursuit of sport of the person 1 can be drawn. This applies in particular where the pressure detection sensors 4 are embodied multiply and arranged at positions of the sports boots 2 that are arranged so as to be distributed, as is known from AT517933B1 by the applicant. In particular, by means of the pressure detection sensors 4 it can be detected in the course of the pursuit of ski sport which weight distribution, dynamics, impulsiveness and force distribution are exerted between the person exercising sports 1 and the respective sports equipment, in particular a pair of skis and/or the respective underground in each case and/or is given with respect to time.

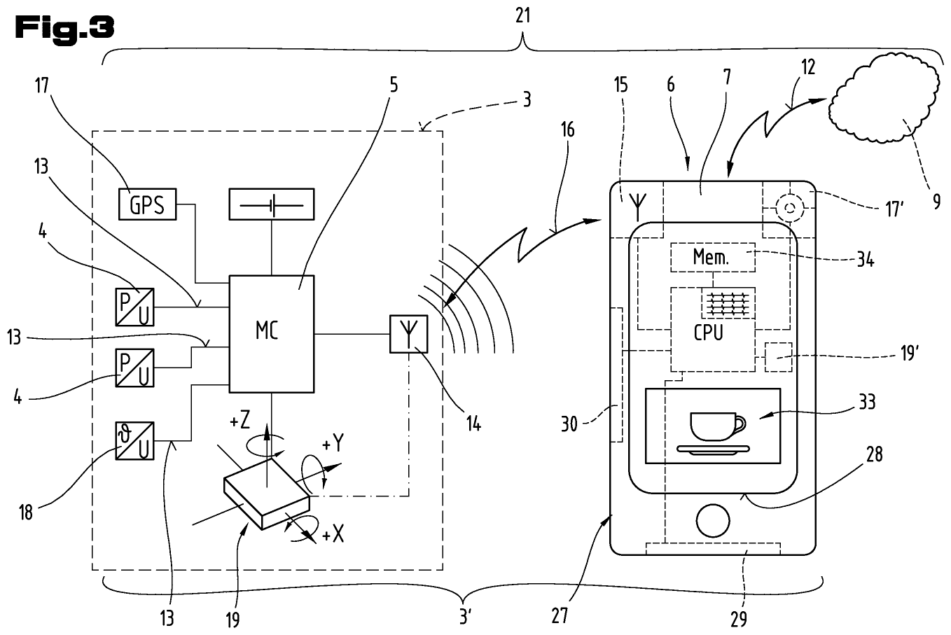

In addition to pressure-sensitive sensors 4, a plurality of further sensors for data-technical detection of the use behavior and/or of the athletic performance ability of the person 1 can be formed and/or arranged on the sports boot 2 and/or on the respective sports equipment and/or directly on the person 1. As can also and best be seen from FIG. 3, the electronic detection system 3, 3′ can also comprise sensors and/or detection systems selected from the following group: acceleration sensors, local and/or global position detection systems 17, 17′, temperature sensors 18, gyroscopic sensors 19, contact detecting sensors and/or switches, speed sensors and timers. By evaluating and correlating the respective sensor signals and/or detection systems, a comprehensive representation of the use behavior and/or current athletic performance ability or skill values 20 of the respective person exercising sports 1 can be gained (FIG. 2).

In particular, it can be provided for that the electronic detection system 3′ reverts to sensor technology and/or incorporates sensor systems that are implemented in the mobile terminal 6, in particular in the smartphone 7, as a standard and are selected from a group comprising local and/or global position detection systems 17′, gyroscopic sensors 19′, inertial measurement units (IMU), acceleration sensors and inclination sensors, as can be schematically seen from FIG. 3. Thereby, extensive capturing and/or detection possibilities are provided, which are based on tested sensor technologies that provide a high functional reliability and can moreover be used and/or jointly used relatively cost-effectively.

In this respect, the performance ability or skill values 20 can comprise parameters selected from a group comprising equilibrium behavior (balance), edging angle of the skis, dynamics of the changes of direction, speed profile, force relationships between the person 1 and the sports boot 2, weight and/or load distribution between left and right sports boot 2, and the like. The performance ability or skill values 20, in particular the current state information or actual data regarding the person exercising sports 1, provided and/or detected by the electronic detection system 3, 3′ are then assessed and/or evaluated by a computer-implemented evaluation means 21. The computer-implemented evaluation means 21 can be defined by a single computer unit or by an interconnection and/or a plurality of correspondingly programmed computer units communicating with one another. It is useful if this computer-implemented evaluation means 21 on the one hand comprises the mobile terminal 6 and on the other hand a peripheral computer system 10, in particular a dedicated server for the presented evaluation system (FIG. 1). Program-technical means (software-technical applications) are implemented and can be executed on the mobile terminal 6, in particular on the smartphone 7. Analogously to this, corresponding program-technical means, in particular software-technically realized operating and evaluation programs, are implemented and can be executed on the computer system 10, in particular on the dedicated server of the system driver, preferably accessible via Internet 9 and/or communicating.

By means of the computer-implemented evaluation means 21, which can be operated by software or program control, —FIG. 1-3—a fully automated ascertainment or at least a semi automated determination of a current level of performance ability or skill of the system user and/or the person 1 exercising sports equipped with the electronic detection system 3, 3′ is allowed for.

The computer-implemented evaluation means 21 is further designed for carrying out a selection or preselection of suitable instructional material 11 for the respective person 1 exercising sports, in particular of for example video-based training material. For this purpose—as is illustrated in FIG. 2—a fully or semi automated selection or preselection step 22 is carried out. Said step can be realized by means of procedures known from the prior art, in particular by assessment and selection procedures. In this regard, in particular the respective determined performance ability or skill values 20 of the person 1 that are as current as possible are included in these assessment and/or selection operations. For this purpose—as is illustrated in FIG. 2—a program-technically implemented weighting and/or merging step 23 is carried out. In this weighting and/or merging step 23, the respective skill and/or performance data of the person 1 is program-technically processed and/or used. The fully automated or semi automated selection step and/or the fully automated or semi automated preselection step 22, which is entirely or at least partially executable by means of the computer-implemented evaluation means 21, is carried out taking into consideration or including the automatedly or semi automatedly determined level of performance ability or skill of the person 1, in particular with automated consideration and evaluation of the current and or last valid performance ability or skill values 20 of the respective person 1.

In this regard, the corresponding selection or preselection process is carried out within a defined, relatively comprehensive instructional material volume 11. The selection step 22 can be realized fully automatedly by the computer-implemented evaluation means 21 and/or by the electronic detection system 3, 3′, meaning that it can be carried out without considerable interaction by the person 1 exercising sports or by another person. In the alternative or in combination to this, a preselection step 22 is conceivable, which ultimately leads to a selection of suitable instructional material 11 and/or instructional material 11 considered adequate taking into account the input of the person 1 exercising sports or of another third person.

The entirety of teaching materials 11 available for a selection may comprise videos 24 (video-based instructional material 11) and/or audio books 25 (sound-based instructional material 11) and/or information documents 26 (text-based and/or image-based instructional material 11). The instructional material(s) 11 which is considered most suitable and/or most helpful in view of the respective performance ability or skill values 20 and is ultimately selected by the evaluation means 21 is then recommended to the person 1 exercising sports and/or to another third person and can then or later be output on the mobile terminal 6 portable by the person 1. This output can be control-technically initiated and can ultimately be released and/or activated by person 1. In the alternative or in combination to this, it can be provided for that the corresponding output is carried out fully automatedly, i.e. without a previous user interaction. In particular, it can be provided for that after a corresponding recommendation by the evaluation means 21, which is preferably output via the mobile terminal 6, an explicit starting command for output of the corresponding instructional material 11 must be entered into the mobile terminal 6.

Typically, dozens, hundreds or even thousands of information and/or data objects in connection with relevant or discipline-specific instructional materials 11 are available. In this regard, the computer-implemented evaluation means 21 is adapted for making a suitable selection or preselection from a high number of generally available instructional materials 11 and to hence provide the person 1 exercising sports with assistance relatively quickly and in a targeted manner. It is also particularly useful in this regard that this assistance in the form of instructional material 11 that is as adequate as possible can be provided relatively promptly, in particular immediately after a pursuit of sports and/or a short sports sequence, or can be provided during the pursuit of sports. The availability of the respective best supporting training or instructional material 11 is almost entirely independent from the respective location of the person 1 exercising sports if sufficiently stable and powerful data communication connections are present.

The output of the respective instructional material 11 is carried out on an output device 27 implemented in accordance with the standard of the mobile terminal 6. Such an output device 27 can comprise a display 28, a speaker 29 and/or a vibration generator 30 (FIG. 3).

In particular, the mobile terminal 6 is adapted for displaying video images and/or text-based comments, in particular text instructions, on its display 28 and further for carrying out an output of sound signals and/or voice instructions by means of the speaker 29. Thereby, the information content and/or learning success and learning progress for the person 1 using the specified electronic system can be increased significantly.

In this regard, the fully or semi automated selection or preselection of instructional material 11 by means of the computer-implemented evaluation means 21 is carried out with a view to improving and/or stabilizing current athletic performance ability or skill values 20 of the user of the specified system, in particular of the person 1 exercising sports.

Furthermore, program-technical means, which allow for retrieval of instructional material 11 (download) via the data and/or communication interface 8 of the portable, mobile terminal 6, can be executed on the mobile terminal 6. In particular, this data and/or communication interface 8 is designed for remote transmission and remote reception of data to and from at least one host computer 31 associated with the system or a corresponding Internet portal 32—FIG. 1. In particular, access to a host computer 31 associated with the system can be gained via the Internet data connection 12 described above. In this regard, the data and/or communication interface 8 of the mobile terminal 6 can be formed by an interface according to the WLAN standard and/or by an interface according to a mobile radio network standard with a sufficient data transmission rate.

According to a useful measure, the mobile terminal 6 can be adapted for carrying out a representational overlay of graphics, symbols or information texts with respect to instructional material 11 to be output, in particular with respect to image or video material 24. This representational overlay is intended for achieving a targeted assistance for athletic improvement of the person 1 depending on automatedly determined execution deficits of the person 1 exercising sports to be supported.

Moreover, the mobile terminal 6 and/or a program-technical means that can be executed thereon can be adapted for carrying out a computer-implemented determination and output of a break recommendation 33. This break recommendation 33 can be carried out by activation of at least one of the output devices 27, for example be issued by actuating the display 28 and/or the speaker 29 and/or the vibration generator 30. In the alternative or in combination to determining and outputting a break recommendation 33, determining and outputting a fatigue quantification by means of the portable mobile terminal 6 can be provided for. In this regard, the results of the break recommendation 33 and/or the fatigue quantification are based on the values detected by the electronic detection system 3, 3′ and/or on the insights gained and/or parameters recorded regarding the person 1 exercising sports by the computer-implemented detection means 21.

In particular, interactions between the person 1 and the presented technical system can be initiated by outputting visually, acoustically and/or tactilely perceptible signals, i.e. be carried out by means of the display 28, the speaker 29, and/or the vibration generator 30 of the portable, mobile terminal 6. In a computer-implemented determination of a fatigue quantification and/or break recommendation 33 for the person 1 exercising sports, the following parameters selected from the group comprising pursuit duration, speed profile, acceleration profile, force profile, sports equipment edging angle, weather conditions such as cold, warm, fog, snowfall and the like, piste conditions such as planar, bumpy, deep snow, hard, soft, icy, grippy snow and the like can be taken into account.

The specified electronic system can also be adapted for storing detected performance ability or skill values 20 and/or determined levels of performance ability or skill in a local storage device 34—FIG. 3—of the portable mobile terminal 6 and/or in an external remotely accessible storage device 35—FIG. 1.

The computer-implemented evaluation means 21 and/or the electronic detection system 3, 3′, in particular in connection with program-technical and/or software-based means (stored program code) executable thereon, can also be adapted for comparing preceding performance ability or skill values 20 stored in the storage device 34 and/or 35 and/or historically determined levels of performance ability or skill with current performance ability or skill values and/or levels of performance ability or skill. Hence, trend analyses can be generated and athletic improvements and/or stabilizations can be achieved with regard to competitive incentives or with regard to improvement incentives of one's own accord. In particular, the self-motivation of a person 1 exercising sports using the specified system can thus be increased.

According to a useful embodiment, the computer-implemented evaluation means 21 can also be adapted for carrying out a comparison of performance ability or skill values and/or of determined levels of performance ability or skill regarding individual, several or all persons belonging to a predefined comparison group. In particular, it can be provided for that via the computer-implemented evaluation means 21, a group of persons is formed and/or can be defined which is then managed as community and/or so-called comparison group. Hence, a competition can be carried out and/or a competition effect can be generated and an improvement of the athletic performance ability of the participants of this group of persons can be achieved.

As elucidated above, the portable mobile terminal 6 assigned to the person 1 exercising sports can fulfill multiple functions. In particular, it can on the one hand be a part of the electronic detection system 3, 3′ and on the other hand be a part of the computer-implemented evaluation means 21, as can also be gathered from FIGS. 1 and 3. In this regard, the portable mobile terminal 6 can be understood as a wearable computer and is typically designed as a smartphone 7. In the alternative or in combination to a smartphone 7, the portable mobile terminal 6 can also be defined by a smartwatch, by smartglasses (data glasses), by a personal electronic assistant or the like.

The exemplary embodiments show possible embodiment variants, and it should be noted in this respect that the invention is not restricted to these particular illustrated embodiment variants of it, but that rather also various combinations of the individual embodiment variants are possible and that this possibility of variation owing to the teaching for technical action provided by the present invention lies within the ability of the person skilled in the art in this technical field.

The scope of protection is determined by the claims. However, the description and the drawings are to be adduced for construing the claims. Individual features or feature combinations from the different exemplary embodiments shown and described may represent independent inventive solutions. The object underlying the independent inventive solutions may be gathered from the description.

Finally, as a matter of form, it should be noted that for ease of understanding of the structure, elements are partially not depicted to scale and/or are enlarged and/or are reduced in size.

Although only a few embodiments of the present invention have been shown and described, it is to be understood that many changes and modifications may be made thereunto without departing from the spirit and scope of the invention.

### LIST OF REFERENCE NUMBERS

1 person
2 sports boots
3, 3′ electronic detection system
4 pressure detection sensors
5 electronic evaluation unit
6 mobile terminal
7 smartphone
8 first communication interface
9 Internet
10 computer system
11 instructional material
12 Internet data connection
13 electrical line links
14 close-range communication interface
15 close-range communication interface
16 close-range communication path
17, 17′ position detecting system
18 temperature sensor
19, 19′ gyroscopic sensor
20 performance ability or skill values
21 computer-implemented evaluation means
22 selection or preselection step
23 weighting and/or merging step
24 video
25 audio book
26 information document
27 output device
28 display
29 speaker
30 vibration generator
31 host computer
32 Internet portal
33 break recommendation
34 storage device (local)
35 storage device (remotely accessible)

1: A method for supporting a person (1) in the pursuit of ski sport, comprising the steps:
detecting the current athletic performance ability or skill values (20) of the person exercising sports by means of an electronic detection system (3, 3′), said electronic detection system (3, 3′) comprising a mobile terminal (6), in particular a smartphone (7), portable by the person (1);
evaluating the condition information or actual data provided by the electronic detection system (3, 3′) by means of a computer-implemented evaluation means (21);
fully automated ascertainment or semi automated determination of the current level of performance ability or skill of the person (1) by means of the computer-implemented evaluation means (21);
selection or preselection of instructional material (11) by means of the computer-implemented evaluation means (21) from a plurality of instructional materials (11) accessible or available to the computer-implemented evaluation means (21), wherein this selection or preselection is carried out by the computer-implemented evaluation means (21) taking into consideration or account the level of performance ability or skill of the person (1) determined automatedly or semi automatedly; and
outputting selected instructional material (11) on the mobile terminal (6) portable by the person (1) exercising sports.

2: The method according to claim 1, further comprising automated selection or preselection of for example video-based instructional material (11), said instructional material (11) being provided for improving the current athletic performance ability or skill values (20).

3: The method according to by claim 1, further comprising a display of video images and/or text-based comments on a display (28) of the portable mobile terminal (6) and/or by an output of voice instructions by means of a speaker (29) of the portable mobile terminal (6).

4: The method according to claim 1, further comprising retrieval of video-based instructional material (11) via a first data or communication interface (8) of the portable mobile terminal (6), said data or communication interface (8) being adapted for remote transmission and remote reception of data in relation to at least one host computer (31) or Internet portal (32).

5: The method according to claim 1, further comprising a representational overlay of graphics, symbols or information texts with respect to video-based instructional material (11) subject to automatedly determined execution deficits of the person (1) to be supported.

6: The method according to claim 1, further comprising computer-implemented detection and output of a fatigue quantification and/or break recommendation (33) on the mobile terminal (6).

7: The method according to claim 6, further comprising the output of visually, acoustically or tactilely perceptible signals by means of the display (28), the speaker (29) and/or the vibration generator (30) of the portable mobile terminal (6).

8: The method according to claim 6, further comprising inclusion of parameters selected from the group consisting of pursuit duration, speed profile, acceleration profile, force profile, sports equipment edging angle, ski style, weather conditions, piste conditions and piste difficulty levels for the computer-implemented determination of a fatigue quantification and/or break recommendation (33).

9: The method according to claim 1, further comprising storing of detected performance ability or skill values (20) and/or of determined levels of performance ability or skill in a local storage device (34) of the portable mobile terminal (6) and/or in an external remotely accessible storage device (35).

10: The method according to claim 1, further comprising comparing temporally preceding performance ability or skill values (20) and/or determined levels of performance ability or skill stored in a storage device (34; 35) to current performance ability or skill values (20) and/or determined levels of performance ability or skill by means of the computer-implemented evaluation means (21).

11: The method according to claim 1, further comprising comparing performance ability or skill values (20) and/or determined levels of performance ability or skill with respect to individual or several persons belonging to a predefined comparison group.

12: The method according to claim 1, further comprising the evaluation of pressure signals from at least one pressure detection sensor (4) by means of at least one electronic evaluation unit (5), said at least one pressure detection sensor (4) being arranged in or on a sports boot (2) that can be worn by the person (1).

13: An electronic system for supporting a person (1) in the pursuit of ski sport, comprising
an electronic detection system (3, 3′) adapted for detecting the current athletic performance ability or skill values (20) of the person exercising sports, said electronic detection system (3, 3′) comprising a mobile terminal (6), in particular a smartphone (7), portable by the person (1);
a computer-implemented evaluation means (21) adapted for evaluating the condition information or actual data provided by the electronic detection system (3, 3′);
wherein the computer-implemented evaluation means (21) is further adapted for the fully automated ascertainment or semi automated determination of the current level of performance ability or skill of the person (1);
and wherein the computer-implemented evaluation means (21) is further adapted for the selection or preselection of instructional material (11) from a plurality of instructional materials (11) accessible or available to the computer-implemented evaluation means (21), wherein this selection or preselection is carried out by the computer-implemented evaluation means (21) taking into consideration or account the level of performance ability or skill of the person (1) determined automatedly or semi automatedly;
and wherein the portable mobile terminal (6) of the person (1) exercising sports is adapted for outputting selected instructional material (11).

14: The electronic system according to claim 13, further comprising at least one pressure detection sensor (4) in or on a sports boot (2) that can be worn by the person (1) and at least one electronic evaluation unit (5) for the pressure signals of the at least one pressure detection sensor (4).

15: The electronic system according to claim 13 with means adapted for carrying out a method for supporting a person (1) in the pursuit of ski sport, comprising the steps:
detecting the current athletic performance ability or skill values (20) of the person exercising sports by means of an electronic detection system (3, 3′), said electronic detection system (3, 3′) comprising a mobile terminal (6), in particular a smartphone (7), portable by the person (1);
evaluating the condition information or actual data provided by the electronic detection system (3, 3′) by means of a computer-implemented evaluation means (21);
fully automated ascertainment or semi automated determination of the current level of performance ability or skill of the person (1) by means of the computer-implemented evaluation means (21):
selection or preselection of instructional material (11) by means of the computer-implemented evaluation means (21) from a plurality of instructional materials (11) accessible or available to the computer-implemented evaluation means (21), wherein this selection or preselection is carried out by the computer-implemented evaluation means (21) taking into consideration or account the level of performance ability or skill of the person (1) determined automatedly or semi automatedly; and
outputting selected instructional material (11) on the mobile terminal (6) portable by the person (1) exercising sports.
